(19) United States
(12) Patent Application Publication
(10) Pub. No.: US 2025/0182612 A1
(43) Pub. Date: Jun. 5, 2025

(54) SYSTEMS AND METHODS FOR PROVIDING ASSISTANCE TO HEARING-IMPAIRED PEDESTRIANS

(71) Applicant: Toyota Motor Engineering & Manufacturing North America, Inc., Plano, TX (US)

(72) Inventors: Benjamin Piya Austin, Saline, MI (US); Rohit Gupta, Santa Clara, CA (US); Rebecca L. Kirschweng, Bloomfield Hills, MI (US); William Patrick Garrett, Plymouth, MI (US); Phillip J. Babian, Canton, MI (US)

(73) Assignees: Toyota Motor Engineering & Manufacturing North America, Inc., Plano, TX (US); Toyota Jidosha Kabushiki Kaisha, Toyota-shi Aichi-ken (JP)

(21) Appl. No.: 18/524,117

(22) Filed: Nov. 30, 2023

(51) Int. Cl. G08G 1/005; A61B 5/12

(52) U.S. Cl. CPC G08G 1/005; A61B 5/123

(57) ABSTRACT

Systems, methods, and other embodiments described herein relate to providing navigation assistance to pedestrians experiencing temporary hearing impairment. In one embodiment, a method includes inferring that a pedestrian is experiencing hearing impairment based on data collected by a user device of the pedestrian. Responsive to an inference of hearing impairment, a hearing test is administered to verify hearing impairment of the pedestrian. A pedestrian assistance countermeasure is produced responsive to verified hearing impairment of the pedestrian as determined from the hearing test.

### TECHNICAL FIELD

The subject matter described herein relates, in general, to ensuring safe vehicle-pedestrian interactions and, more particularly, to assisting pedestrians who may be experiencing temporary or long-term hearing impairment.

### BACKGROUND

Vehicle roadways and the adjacent infrastructure are becoming increasingly complex and populated with motorists and pedestrians. This is perhaps most apparent in urban areas with significant population and vehicle densities. As both vehicles and pedestrians are regularly near one another based on their respective use of roadways and adjacent infrastructure elements (e.g., sidewalks) and the occasional occupation of the roadways by pedestrians (such as at crosswalks), vehicle-pedestrian interactions are inevitable and a regular occurrence. For example, a pedestrian may desire to cross a road to reach an intended destination. Pedestrians generally use crosswalks to traverse the road to reach their destination safely.

Some factors may negatively impact the safety of such pedestrian-vehicle interactions. For example, pedestrians with hearing impairments may face challenges when navigating a busy roadway environment, as they may not hear warning signals such as car horns or emergency sirens. Pedestrians with hearing impairment may also have difficulty communicating with others, especially in noisy environments. Additionally, pedestrians with hearing impairment may have difficulty identifying the direction and distance of sounds, making it harder to locate a noise/sound source. As such, a pedestrian with hearing impairment increases the risk of a potentially dangerous pedestrian-vehicle interaction.

Hearing impairment may result from any number of circumstances. For example, a pedestrian may be adjacent to a construction site where a loud jackhammer has temporarily impaired their hearing. The pedestrian may recognize the loud noise but may be unaware of the extent to which their hearing is impaired and the potential danger that may be caused by said hearing impairment.

### SUMMARY

In one embodiment, example systems and methods relate to a manner of improving pedestrian safety when navigating busy roadway environments.

In one embodiment, an impaired hearing detection system for assisting pedestrians with hearing impairment is disclosed. The impaired hearing detection system includes one or more processors and a memory communicably coupled to the one or more processors. The memory stores instructions that, when administered by the one or more processors, cause the one or more processors to infer that a pedestrian is experiencing hearing impairment based on data collected by a user device of the pedestrian. The memory also stores instructions that, when administered by the one or more processors, cause the one or more processors to administer a hearing test to verify the hearing impairment of the pedestrian responsive to an inference of hearing impairment. The memory also stores instructions that, when administered by the one or more processors, cause the one or more processors to produce a pedestrian assistance countermeasure responsive to the verified hearing impairment of the pedestrian as determined from the hearing test.

In one embodiment, a non-transitory computer-readable medium for assisting pedestrians with hearing impairment and including instructions that, when administered by one or more processors, cause the one or more processors to perform one or more functions is disclosed. The instructions include instructions to infer that a pedestrian is experiencing hearing impairment based on data collected by a user device of the pedestrian. The instructions also include instructions to administer a hearing test to verify the hearing impairment of the pedestrian responsive to an inference of hearing impairment. The instructions also include instructions to produce a pedestrian assistance countermeasure responsive to the verified hearing impairment of the pedestrian as determined from the hearing test.

In one embodiment, a method for assisting pedestrians with hearing impairment is disclosed. In one embodiment, the method includes inferring that a pedestrian is experiencing hearing impairment based on data collected by a user device of the pedestrian. The method also includes executing a hearing test to verify the hearing impairment of the pedestrian responsive to an inference of hearing impairment. The method also includes producing a pedestrian assistance countermeasure responsive to the verified hearing impairment of the pedestrian as determined from the hearing test.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 illustrates one embodiment of an impaired hearing detection system that is associated with assisting a pedestrian who is experiencing hearing impairment.

FIG. 2 depicts the impaired hearing detection system aiding a pedestrian who is experiencing hearing impairment.

FIG. 3 depicts the impaired hearing detection system inferring hearing impairment based on the conversation data of the pedestrian and another conversant in a noisy environment.

FIG. 4 illustrates one embodiment of the impaired hearing detection system of FIG. 1 in a cloud-computing environment.

FIG. 5 illustrates one embodiment of a machine-learning impaired hearing detection system associated with assisting pedestrians exhibiting impaired decision-making.

FIG. 6 illustrates a flowchart for one embodiment of a method associated with assisting pedestrians exhibiting impaired hearing.

### DETAILED DESCRIPTION

Systems, methods, and other embodiments associated with improving pedestrian safety while navigating busy roadways or other environments where enhanced pedestrian perception increases pedestrian safety are disclosed herein. As previously described, pedestrians regularly interact with motor vehicles, for example, on busy streets and intersections. While typically involving a degree of risk to a pedestrian, these environments can be navigated safely. Such navigation, however, relies on an accurate perception of the environment, including the sound environment. For example, an emergency siren or car horn is an audible signal to warn pedestrians and other motorists of a situation that may dictate increased attention. If pedestrians cannot perceive the sound environment, they may be unaware of the audible cues intended to protect them.

Moreover, in some examples, a pedestrian is unaware of the extent of their hearing impairment and/or the negative implications of their impaired hearing. For example, there may be a scenario where a pedestrian has just walked past a construction site in which a loud jackhammer has temporarily impaired their hearing. The pedestrian may recognize the loud noise but may be unaware of the extent to which their hearing is impaired and the potential danger that may be caused by said hearing impairment. That is to say, a pedestrian may not be aware that their hearing impairment places them in a potentially dangerous situation.

Furthermore, to ensure the safety of the pedestrians and others that utilize the roadways and adjacent infrastructure, drivers of vehicles may need to exercise additional caution. However, it may be the case that such drivers cannot ascertain the impaired hearing state of pedestrians. Some vehicles are semi-autonomous or fully autonomous, where at least part of the control of the vehicle is handed over from the driver to autonomous control systems. These autonomous control systems, if not aware of hearing-impaired pedestrians, may not be able to control the vehicle in such a way as to prevent or reduce the likelihood of a dangerous interaction with the pedestrian.

As such, the present impaired hearing detection system identifies a pedestrian experiencing hearing impairment and provides countermeasures to reduce the likelihood of potentially dangerous conditions that may result were the pedestrian to remain in a hearing-impaired state without hearing audible cues that promote their safety. The system may implement a multi-stage hearing evaluation operation. First, the system infers whether the pedestrian is likely experiencing hearing impairment. As a specific example of the first stage, the system detects loud sounds using the microphone of the mobile device. If the detected decibel level is above a threshold, the system can infer the pedestrian is experiencing hearing impairment.

In another example, the system passively tests the pedestrian's hearing based on the behaviors of the pedestrian while communicating via a user device. Conversational indicators of hearing impairment include 1) the pedestrian talking louder than the pedestrian's usual volume, 2) the pedestrian asking participants in the conversation to repeat themselves more frequently than usual, 3) the pedestrian repeating themselves, 4) the sharpness of the pedestrian's words diminishing, and 5) the pedestrian elongating their words, among others. In an example, based on the context of the conversation, the system can differentiate between 1) a pedestrian experiencing hearing impairment and 2) a pedestrian who does not understand what someone is saying. For example, if the person the pedestrian is talking to is talking quietly or quickly, the pedestrian may not be experiencing hearing impairments. The system can also infer the pedestrian's hearing state based on the gait of the pedestrian. For example, if after a loud noise is heard, the pedestrian increases their step length, it may be that the pedestrian is experiencing hearing impairments and is trying to catch their footing.

In any event, the system verifies the inference by administering a hearing test to the pedestrian. In an example, the hearing test is administered by providing the pedestrian with a low, quiet tone or an array of tones that vary in frequency and volume. If the pedestrian hears a tone with a threshold frequency and/or volume, the system may infer that the pedestrian has sufficient hearing to navigate an environment safely. By comparison, if the pedestrian does not hear the tone having the threshold frequency and/or volume, the system may infer that the pedestrian has a hearing impairment to a degree that a countermeasure should be provided. As such, the threshold may be a tone/frequency that, if not heard, could put the pedestrian at risk and/or compromise the pedestrian's safety. Various countermeasures may be provided. As an example, the system can suggest that the pedestrian wear headphones, put in hearing aids, consult a physician, move away from an area with a high noise level or heavy traffic, or remain stationary until their hearing is restored.

In this way, the disclosed systems, methods, and other embodiments improve pedestrian safety by providing notifications and recommendations to a pedestrian based on a detected impaired hearing state. The disclosed systems, methods, and other embodiments also improve vehicle functionality by apprising vehicle drivers and autonomous vehicle systems of the presence of hearing-impaired pedestrians and, in some cases, improve vehicle control by controlling the vehicle in response to a detected pedestrian with hearing impairment.

As such, the impaired hearing detection system reduces the likelihood of potentially dangerous situations created by pedestrians who are experiencing hearing impairment but who are unaware of the severity of their hearing impairment and/or do not appreciate the effect hearing impairment has on safety. As such, the present systems, methods, and other embodiments recognize hearing-impaired pedestrians, notify the pedestrian of the impairments, and provide recommendations/controls that alleviate the adverse side effects of impaired hearing.

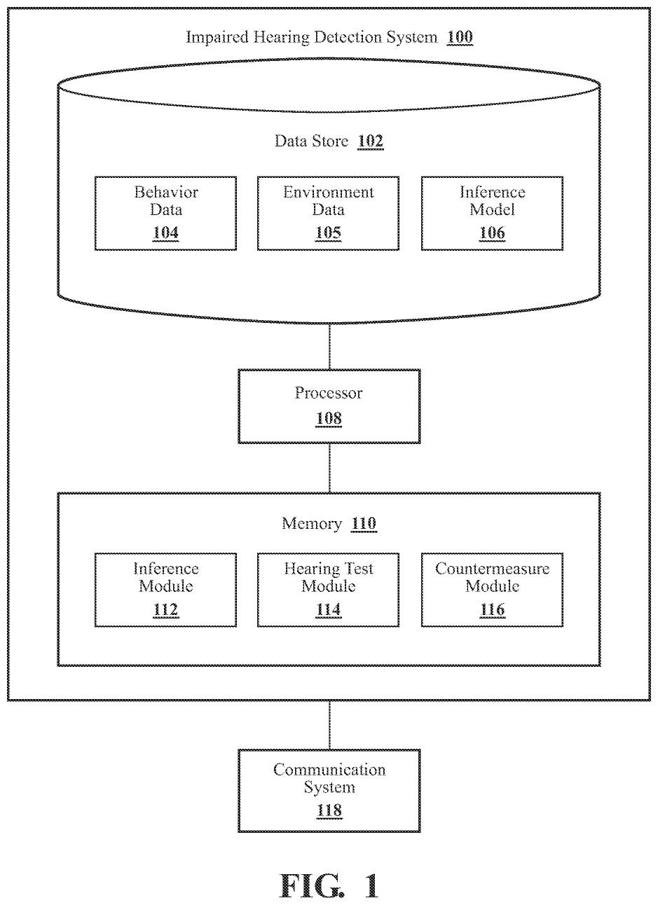

Turning now to the figures, FIG. 1 illustrates one embodiment of an impaired hearing detection system 100 that is associated with assisting pedestrians exhibiting impaired hearing. It will be appreciated that for simplicity and clarity of illustration, where appropriate, reference numerals have been repeated among the different figures to indicate corresponding or analogous elements. In addition, the discussion outlines numerous specific details to provide a thorough understanding of the embodiments described herein. Those of skill in the art, however, will understand that the embodiments described herein may be practiced using various combinations of these elements. In any case, the impaired hearing detection system 100 is implemented to perform methods and other functions as disclosed herein relating to improving pedestrian safety, even when the pedestrian is exhibiting impaired decision-making.

With reference to FIG. 1, one embodiment of the impaired hearing detection system 100 is illustrated. The impaired hearing detection system 100 is shown as including a processor 108. In one or more arrangements, the processor(s) 108 can be a primary/centralized processor of the impaired hearing detection system 100 or may be representative of many distributed processing units. For instance, the processor(s) 108 can be an electronic control unit (ECU). Alternatively, or additionally, the processor(s) 108 include a central processing unit (CPU), an ASIC, a microcontroller, a system on a chip (SoC), and/or other electronic processing unit. As will be discussed in greater detail subsequently, the impaired hearing detection system 100, in various embodiments, may be implemented as a cloud-based service.

In one embodiment, the impaired hearing detection system 100 includes a memory 110 that stores an inference module 112, a hearing test module 114, and a countermeasure module 116. The memory 110 is a random-access memory (RAM), read-only memory (ROM), a hard-disk drive, a flash memory, or another suitable memory for storing the modules 112, 114, and 116. In alternative arrangements, the modules 112, 114, and 116 are independent elements from the memory 110 that are, for example, comprised of hardware elements. Thus, the modules 112, 114, and 116 are alternatively ASICs, hardware-based controllers, a composition of logic gates, or another hardware-based solution.

In at least one arrangement, the modules 112, 114, and 116 are implemented as non-transitory computer-readable instructions that, when executed by the processor 108, implement one or more of the various functions described herein. In various arrangements, one or more of the modules 112, 114, and 116 are a component of the processor(s) 108, or one or more of the modules 112, 114, and 116 are administered on and/or distributed among other processing systems to which the processor(s) 108 is operatively connected.

Alternatively, or in addition, the one or more modules 112, 114, and 116 are implemented, at least partially, within hardware. For example, the one or more modules 112, 114, and 116 may be comprised of a combination of logic gates (e.g., metal-oxide-semiconductor field-effect transistors (MOSFETs)) arranged to achieve the described functions, an application-specific integrated circuit (ASIC), programmable logic array (PLA), field-programmable gate array (FPGA), and/or another electronic hardware-based implementation to implement the described functions. Further, in one or more arrangements, one or more of the modules 112, 114, and 116 can be distributed among a plurality of the modules described herein. In one or more arrangements, two or more of the modules described herein can be combined into a single module.

In one embodiment, the impaired hearing detection system 100 includes the data store 102. The data store 102 is, in one embodiment, an electronic data structure stored in the memory 110 or another data storage device and that is configured with routines that can be executed by the processor 108 for analyzing stored data, providing stored data, organizing stored data, and so on. Thus, in one embodiment, the data store 102 stores data used by the modules 112, 114, and 116 in executing various functions.

The data store 102 can be comprised of volatile and/or non-volatile memory. Examples of memory that may form the data store 102 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, solid-state drivers (SSDs), and/or other non-transitory electronic storage medium. In one configuration, the data store 102 is a component of the processor(s) 108. In general, the data store 102 is operatively connected to the processor(s) 108 for use thereby. The term “operatively connected,” as used throughout this description, can include direct or indirect connections, including connections without direct physical contact.

In one embodiment, the data store 102 stores the behavior data 104 along with, for example, metadata that characterizes various aspects of the behavior data 104. For example, the metadata can include location coordinates (e.g., longitude and latitude), relative map coordinates or tile identifiers, time/date stamps from when the separate behavior data 104 was generated, and so on.

In general, the behavior data 104 is data collected by a user device of the pedestrian, which is indicative of the behavior of the pedestrian. As described above, the behaviors of the pedestrian may be indicative of whether or not the pedestrian is suffering from hearing impairment. For example, a pedestrian covering one car while holding a phone up to the other car may indicate that the pedestrian is on a phone call, having a hard time hearing the conversation, and is trying to block out ambient noise. As another example, a pedestrian with erratic pacing and who is turning their back to a noisy area such as a construction site may indicate that the pedestrian is having a hard time hearing a phone call and is trying to find a location to hear the conversation better. As described above, hearing impairment may negatively impact pedestrian safety for various reasons. As such, the behavior data 104 includes data indicative of a behavior of the pedestrian that is relied on by the inference module 112 to infer that the pedestrian is in an environment where their hearing is impaired.

The behavior data 104 may take a variety of forms. In one example, the behavior data 104 includes conversation data collected by a user device, such as a smartphone, tablet, smartwatch, or other mobile device, of the pedestrian. Specifically, the user device may include a microphone that records verbal communication, such as when a pedestrian is on a phone or video call. During these phone or video calls, the pedestrian speaks with certain communication characteristics that are reflected in the conversation data. Examples of verbal communication characteristics include, but are not limited to, cadence, speed, volume, pitch, pronunciation, fluency, articulation, word choice, use of filler words, and pauses between words/phrases. Other examples include the sharpness of the spoken words/phrases and the elongation of the words/phrases. That is, diminished sharpness of words and increased elongation of words may indicate that the pedestrian is having difficulty hearing. As another example, the actual words used by the pedestrian may indicate hearing impairment. For example, the phrase “can you repeat that?” uttered on a phone call may indicate that the pedestrian is, perhaps temporarily, hearing impaired. As such, the behavior data 104 includes the abovementioned conversation data and other information recorded by a microphone during a phone conversation.

The behavior data 104 may include historical records of conversation data for the pedestrian. That is, a determination regarding whether a pedestrian is hearing impaired may be based, at least in part, on a deviation of current conversational behavior from expected conversational behavior for the pedestrian. For example, a pedestrian may usually speak at a certain rate and with a certain volume. At a particular point in time, the behavior data 104 may include a series of temporally related messages from the pedestrian that is at a higher volume and a slower rate than the baseline rate and volume. This may indicate that the pedestrian is having difficulty hearing and may thus be in a safety-compromised state. As such, the behavior data 104 includes a history of the conversational characteristics of the pedestrian to form a baseline against which current conversation data is compared to determine whether the pedestrian is experiencing hearing impairment.

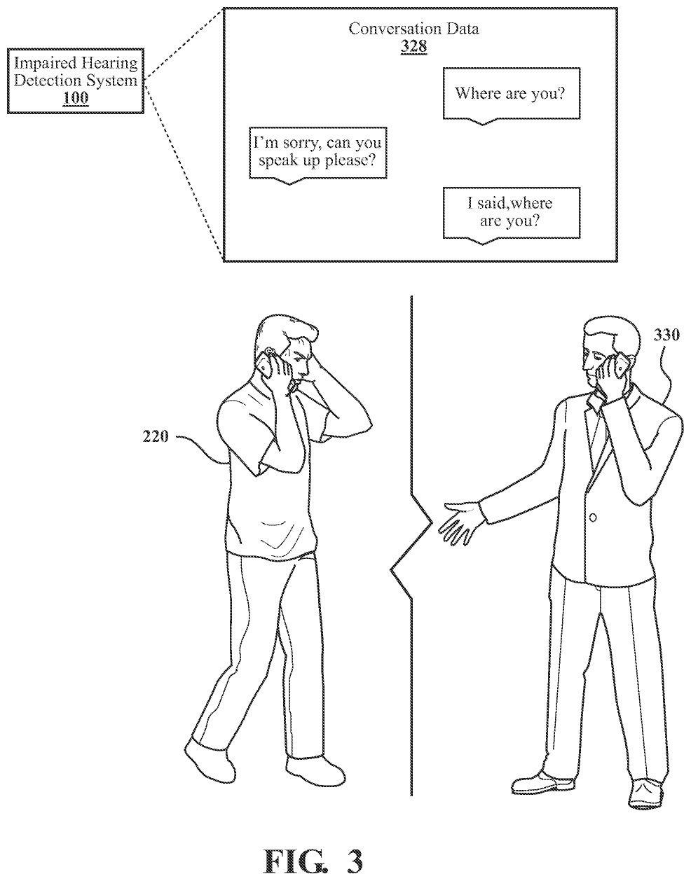

In an example, the behavior data 104 may include conversation data for additional individuals. In one particular example, the additional individual is a participant in a phone conversation with the pedestrian. That is, the conversational characteristics of the pedestrian and the other participant in the conversation may indicate whether the pedestrian is experiencing hearing impairment. For example, a non-pedestrian participant who, throughout a conversation, increases their speaking volume and/or slows their rate of speech may be doing so at the participant's request and may indicate that the pedestrian is having a difficult time hearing the conversation. As another example, the other conversant asking, “can you hear me?” may indicate that the pedestrian has not heard the conversant in the conversation. In this example, the communication characteristics of the other conversant are similarly captured by the microphone of the pedestrian during the conversation. FIG. 3 below depicts an example of pedestrian and conversant conversation data being collected.

As another example, the behavior data 104 includes conversation data collected by other user devices. As described above, the impaired hearing detection system 100 may identify deviations of current conversational characteristics from baseline patterns to identify an impaired decision-making state. As described above, such a comparison may be between current conversational characteristics and baseline conversational patterns for the pedestrian. In another example, such a comparison may be between current conversational characteristics for the pedestrian and baseline conversational patterns for additional users such as a general body of individuals. For example, deviations of the pedestrian's behavior from a general population's communication behavior may provide additional data points by which pedestrian hearing impairment is determined. As such, the behavior data 104 may include conversation data for additional users such that the inference module 112 may infer hearing impairment more accurately based on many data points (e.g., baseline behavior of the pedestrian and baseline behavior of a more general population).

As described above, the behavior data 104 includes a recording of audio collected by a microphone of the user device of the pedestrian. That is, a pedestrian may use the user device to call another individual. In this example, audio recordings may be collected by the user device and transmitted to the impaired hearing detection system 100 via a communication system 118, as described below.

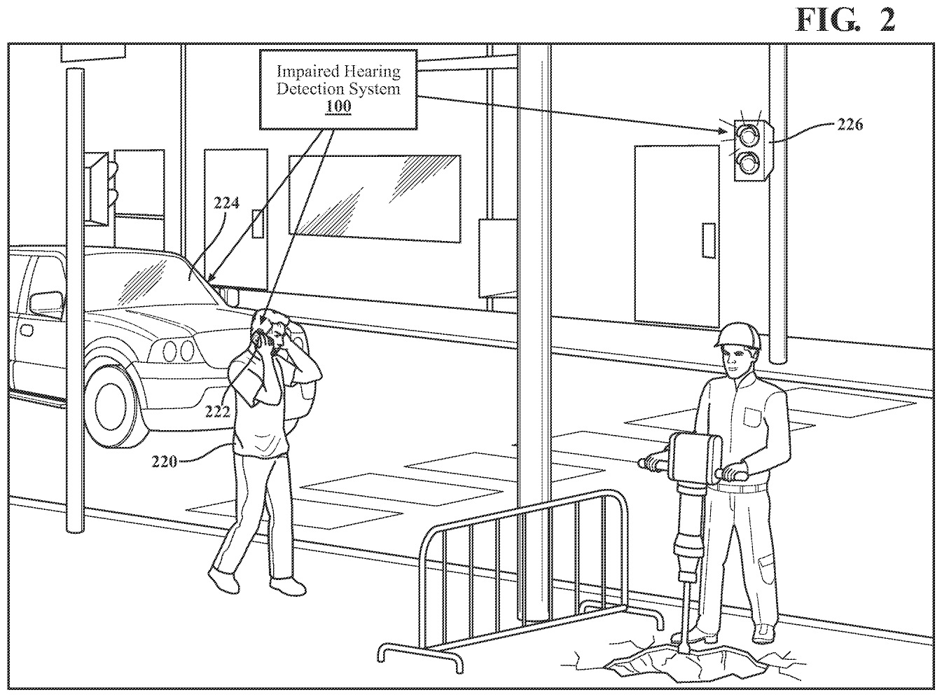

The behavior data 104 also includes movement data. As described above, certain movements may be indicative of pedestrian hearing impairment. For example, a pedestrian moving around in erratic walking patterns in a noisy environment may indicate the pedestrian is trying to find a spot where they can hear a phone call. In one example, a pedestrian's gait may indicate hearing impairment. That is, it may be that when in a noisy environment and/or when the pedestrian is experiencing hearing impairment, a pedestrian increases their step length, for example, to catch their footing. As another example, a pedestrian may bring their hand to the opposite car from where a phone is located, as depicted in FIG. 2, to block out a noisy environment. As another example, the facial expressions and or eye movements of the pedestrian may indicate whether they are having a hard time hearing a phone conversation. As such, the movement data may include data (such as images, accelerometer output, or other sensor output) that indicate the physical movement of the pedestrian as well as the movement of different portions of the pedestrian, such as facial expressions, appendage movement, and eye movement.

As with the conversation data, the behavior data 104 may include historical movement data for the pedestrian and/or other individuals, which historical movement data serves as a baseline against which currently measured movement data is compared to identify deviations, which may indicate a pedestrian hearing impairment.

As such, the behavior data 104 includes movement data, which may be relied on by the inference module 112 in inferring whether or not the pedestrian is experiencing hearing impairment. As with the conversation data, the movement data may be received via a pedestrian user device via a communication system 118.

As described above, the behavior data 104 is collected from pedestrian user devices. Such data collection components include, but are not limited to, a microphone to collect conversation data and one or more of a global-positioning system (GPS) system, accelerometer, and cameras, among others, to track the movement of the pedestrian and other individuals. In an example, this behavior data 104 may be collected from one or more user devices. For example, a mobile phone may include 1) a microphone for recording conversation data for the pedestrian and another conversant and 2) location and/or movement-based sensors for collecting movement data. In another example, some of this information (e.g., movement data) may be collected by another device, such as a wearable health monitoring device and/or infrastructure elements. For example, a watch of the pedestrian may calculate the pedestrian's movement and a camera of a phone, a watch, or that is mounted to an infrastructure element near the pedestrian may capture the pedestrian's movement, facial expressions, and/or eye positions. In one or more arrangements, the movement sensors include one or more accelerometers, one or more gyroscopes, an inertial measurement unit (IMU), a dead-reckoning system, a global navigation satellite system (GNSS), a global positioning system (GPS), and/or other sensors for monitoring aspects about the pedestrian. Note that while various examples of different types of sensors are described herein, it will be understood that the embodiments are not limited to the particular sensors described.

The impaired hearing detection system 100 includes a communication system 118 that facilitates communication with the devices and infrastructure elements such that the behavior data 104 may be collected and stored. In one embodiment, the communication system 118 communicates according to one or more communication standards. For example, the communication system 118 can include multiple different antennas/transceivers and/or other hardware elements for communicating at different frequencies and according to respective protocols. The communication system 118, in one arrangement, communicates via a communication protocol, such as WiFi, DSRC, V2I, V2V, or another suitable protocol for communicating impaired hearing detection system 100 and user devices. Moreover, the communication system 118, in one arrangement, further communicates according to a protocol, such as a global system for mobile communication (GSM), Enhanced Data Rates for GSM Evolution (EDGE), Long-Term Evolution (LTE), 5G, or another communication technology that provides for the user devices communicating with various remote devices (e.g., a cloud-based server). In any case, the impaired hearing detection system 100 can leverage various wireless communication technologies to provide communications to other entities, such as members of the cloud-computing environment.

In one embodiment, the data store 102 further includes environment data 105. In general, information about the surrounding environment of the pedestrian may be indicative of hearing loss. For example, the pedestrian being found in a loud environment such as a construction site, sporting event, or concert venue supports an inference that the pedestrian is hearing impaired. The environment data 105 includes this contextual data, which indicates hearing impairment.

In an example, the environment data 105 is manually transmitted by a pedestrian. For example, pedestrians may self-report that they are in a noisy environment. In another example, the environment data 105 indicates calendar events for the pedestrian. For example, a calendar of the pedestrian may indicate that the pedestrian is scheduled to attend a sporting event that is likely to be noisy. In another example, the environment data 105 includes location-based information for the pedestrian. For example, the environment data 105 may indicate that the pedestrian is in a concert venue. In any case, the environment data 105 is retrieved from the user device or another device on which it originates via the communication system 118.

The data store 102 further includes an inference model 106, which may be relied on by the inference module 112 to infer whether the pedestrian is hearing impaired. The impaired hearing detection system 100 may be a machine-learning system. A machine-learning system generally identifies patterns and/or deviations based on previously unseen data. In the context of the present application, a machine-learning impaired hearing detection system 100 relies on some form of machine learning, whether supervised, unsupervised, reinforcement, or any other type, to infer whether the pedestrian is experiencing hearing impairment based on the observed behavior (i.e., conversational behavior and/or movement behavior) of the pedestrian. In an example, the inference model 106 is a supervised model where the machine learning is trained with an input data set and optimized to meet a set of specific outputs. In another example, the inference model 106 is an unsupervised model where the model is trained with an input data set but not optimized to meet a set of specific outputs; instead, it is trained to classify based on common characteristics. As another example, the inference model 106 may be a self-trained reinforcement model based on trial and error.

In any case, the inference model 106 includes the weights (including trainable and non-trainable), biases, variables, offset values, algorithms, parameters, and other elements that operate to output an inference of hearing impairment of the pedestrian based on any number of input values including conversational behavior data and movement behavior data. Examples of machine-learning models include, but are not limited to, logistic regression models, Support Vector Machine (SVM) models, naïve Bayes models, decision tree models, linear regression models, k-nearest neighbor models, random forest models, boosting algorithm models, and hierarchical clustering models. While particular models are described herein, the inference model 106 may be of various types intended to classify pedestrians based on determined interaction characteristics.

The impaired hearing detection system 100 further includes an inference module 112 which, in one embodiment, includes instructions that cause the processor 108 to infer that a pedestrian is experiencing hearing impairment based on data collected by a user device of the pedestrian. As described above, a pedestrian may be experiencing impaired hearing for various reasons. Data collected by the pedestrian's user device is analyzed in the first of a multi-stage hearing impairment detection operation. The inference module 112 analyzes the data to infer whether a pedestrian is experiencing hearing impairment, which inference is later verified by subjecting the pedestrian to a hearing test. Given the relationship between hearing impairment and pedestrian safety, determining whether or not a pedestrian is experiencing hearing impairment may lead to increased pedestrian safety.

In an example, the data includes environmental audio data, which may be recorded by a microphone of the pedestrian's user device or another device. That is, the user device may include a microphone or other sound level monitoring device, which continuously or periodically monitors the intensity or loudness of detected sounds. In this example, if a detected sound is greater than a threshold amount (such as 85 decibels (dB) or 95 dB) for greater than a threshold period (e.g., 1 second, 10 seconds, 1 minute, etc.), the inference module 112 may infer that the pedestrian is experiencing hearing impairment based on a correlation between loud noises and hearing impairment.

In an example, the data includes behavior data 104 indicative of the behavior of the pedestrian. That is, the inference module 112 operates to acquire the behavior data 104 from the data store 102 and infers hearing impairment of the pedestrian based on such.

As described above, the behavior data 104 may include conversation data. As such, the inference module 112 may include instructions that cause the processor 108 to infer that the pedestrian is experiencing hearing impairment based on conversation data collected by a microphone of a user device of the pedestrian. As described above, certain verbal communication characteristics are indicative of impaired hearing. As one specific example, a pedestrian who repeatedly uses words like “what,” “I can't hear you,” or who asks a conversant to repeat what they said may be experiencing hearing impairment. These characteristics and others are captured in the behavior data 104 and identified by the inference module 112. That is, the inference module 112 may include a speech analysis component that analyses the conversation data to identify the conversational characteristics indicative of impaired hearing. Note that while particular reference is made to particular verbal communication characteristics, the inference module 112 may rely on other behavior data to infer hearing impairment.

As another example, the behavior data 104 includes movement data. That is, similar to conversational characteristics, certain physical movements of the pedestrian may be indicative of impaired hearing. For example, a pedestrian erratically pacing or walking away from a noisy environment may indicate hearing impairment and the pedestrian's efforts to reduce the background noise. Other physical movements that may be found in the movement data and indicative of hearing impairment include arm/hand gestures and facial and eye movements. As such, the inference module 112 acquires this movement data (e.g., images, etc.) and performs object and/or pose recognition/tracking to determine whether the pedestrian performs movements indicative of hearing loss and infers hearing loss based on such. Accordingly, in one embodiment, the inference module 112 includes instructions that cause the processor 108 to infer that the pedestrian is experiencing hearing impairment based on the physical movements of the pedestrian.

In an example, the inference depends on a deviation of measured behavior characteristics from baseline data, which baseline data may pertain to either the pedestrian or other individuals such as a regional or broad public. As such, the baseline data may include behavior data 104 and associated metadata collected from the user device of the pedestrian and user devices of other users. The baseline data may take various forms and generally reflects the historical patterns (e.g., conversational or movement) of those for whom it is collected. As specific examples, baseline conversation data may include historical verbal patterns of speaking cadence, speaking speed, speaking volume, speaking pitch, speaking pronunciation, speaking fluency, speaking articulation, word choice, use of filler words, grammatical errors, and spacing between words/phrases.

By comparing current behavior data 104 against baseline data, the inference module 112 can infer the state of hearing for the pedestrians. For example, measured conversational characteristics of reduced speaking speed, increased volume, increased spacing between words, and the presence of certain phrases such as, “can you speak up?” and “can you repeat that?” as compared to baseline data for a pedestrian may indicate that the pedestrian is experiencing temporary hearing impairment. As such, a recommended countermeasure should be produced.

In an example, the baseline data may be classified based on metadata associating the baseline data with the states of hearing of the pedestrian and other individuals. Put another way, the baseline data may include baseline data for the pedestrian and other users when hearing is unimpaired and baseline data for the pedestrian and other users when they have been identified as experiencing hearing impairment. For example, measured conversation data may be compared against baseline conversation data when the pedestrian experienced impaired hearing to identify similarities in the data set to determine whether a user is experiencing impaired hearing. By comparison, measured conversation data may be compared against baseline conversation data when the pedestrian is not experiencing impaired hearing to identify deviations in the data set.

As described above, the baseline data may include similar data for a body of users, geospatially related or unrelated to the pedestrian. That is, historical behavior patterns, and in some cases, an associated hearing impairment state, for a general population or a subset of the general population that is in the same region as the pedestrian (i.e., a regional population) may serve as a baseline for comparison of measured behavior data. In other words, the inference module 112, which may be a machine-learning module, identifies behavior patterns in the expected behavior of the pedestrian and/or other users and determines when the pedestrian's current behavior deviates or aligns with those patterns. Those deviations and the characteristics of the deviation (e.g., number of deviations, frequency of deviations, degree of deviations) are relied on in determining whether the pedestrian is likely to be experiencing hearing impairment.

Whatever data is included in the baseline data (e.g., historical patterns of the pedestrian, historical patterns of a broader population, or both), the inference module 112 infers a hearing state of the pedestrian based on deviations from measured interaction characteristics against the baseline data. Specifically, the inference module 112 may include instructions that cause the processor 108 to infer hearing loss based on at least one of 1) a degree of deviation between the behavior data and the baseline data and/or 2) a number of deviations between the behavior data and the baseline data within a period of time. That is, certain deviations from an expected behavior (i.e., the baseline interactions) may not indicate impaired hearing but may be attributed to natural variation or another cause. Accordingly, the inference module 112 may include a deviation threshold against which the deviations are compared to classify the pedestrian's hearing state. Specifically, the inference module 112 may be a machine-learning module that considers the quantity and quantity of deviations over time to infer hearing loss.

As described above, the inference may also be based on environment data 105, which indicates a sound environment around the pedestrian. For example, behavior data 104, indicative of hearing loss, may be less heavily weighted if the pedestrian is not in a loud environment, as the behavior data 104 may indicate another condition. For example, certain phrases and communication habits may indicate impaired hearing or a lack of understanding of a concept discussed. If a pedestrian exhibits certain behaviors in a low-sound environment, it may indicate that the pedestrian is not understanding a concept discussed and does not have a hearing impairment. By comparison, if the pedestrian exhibits the same behaviors in a noisy environment, it may indicate the pedestrian is experiencing hearing loss. It should be noted that the inference module 112 may rely on multiple pieces of data when making an inference. That is, a single detected deviation from baseline, a single observed communication characteristic, or a single environmental condition may not be indicative of hearing impairment. As such, the inference module 112 relies on multiple inputs to infer hearing loss.

As the inference module 112 relies on behavior data 104 and environment data 105 to infer hearing impairment, the inference module 112 generally includes instructions that function to control the processor 108 to receive behavior data 104 and/or environment data 105 from the data store 102. The inference module 112, in one embodiment, controls the respective devices to provide the data inputs in the form of the behavior data 104 and environment data 105.

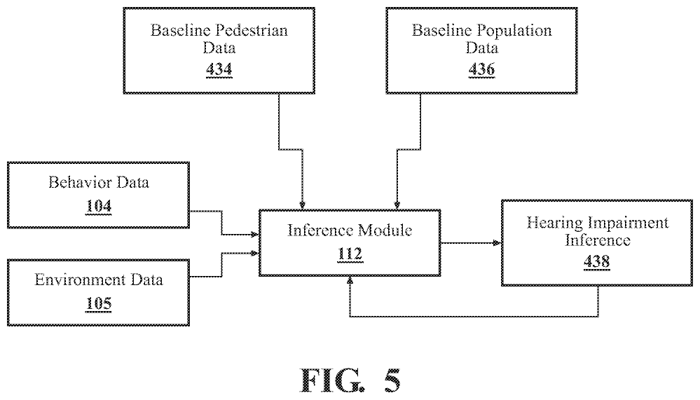

In one approach, the inference module 112 implements and/or otherwise uses a machine learning algorithm. A machine-learning algorithm generally identifies patterns and deviations based on previously unseen data. In the context of the present application, a machine-learning inference module 112 relies on some form of machine learning, whether supervised, unsupervised, reinforcement, or any other type of machine learning, to identify patterns in pedestrian and other individuals' expected behavior and infer whether the pedestrian is experiencing hearing impairment based on 1) the observed behavior data 104, 2) a comparison of the observed behavior data 104 to historical patterns for the pedestrian and/or other users, and/or 3) environment data 105 associated with the behavior data 104. As such, as depicted in FIG. 5, the inputs to the inference module 112 include the behavior data 104 and the environment data 105 for the pedestrian, as well as baseline data for the pedestrian and other individuals. The inference module 112 relies on a mapping between behaviors and impaired hearing, determined from the training set, which includes baseline data, to determine the likelihood of hearing impairment of the pedestrian based on the monitored behaviors of that pedestrian.

In one configuration, the machine learning algorithm is embedded within the inference module 112, such as a convolutional neural network (CNN) or an artificial neural network (ANN) to perform pedestrian classification over the behavior data 104 and environment data 105, from which further information is derived. Of course, in further aspects, the inference module 112 may employ different machine learning algorithms or implement different approaches for performing the hearing impairment inference, which can include logistic regression, a naïve Bayes algorithm, a decision tree, a linear regression algorithm, a k-nearest neighbor algorithm, a random forest algorithm, a boosting algorithm, and a hierarchical clustering algorithm among others to generate pedestrian classifications. Other examples of machine learning algorithms include but are not limited to deep neural networks (DNN), including transformer networks, convolutional neural networks, recurrent neural networks (RNN), Support Vector Machines (SVM), clustering algorithms, Hidden Markov Models, and so on. It should be appreciated that the separate forms of machine learning algorithms may have distinct applications, such as agent modeling, machine perception, and so on.

Whichever particular approach the inference module 112 implements, the inference module 112 improves hearing impairment detection by introducing machine-learning processing of hundreds, thousands, or millions of pieces of data. For example, the inference module 112 may receive information from hundreds, thousands, or tens of thousands of individuals with multiple behaviors that may or may not indicate hearing impairment. Through machine learning, this complex data, which would be impossible to process otherwise, is processed to identify patterns against which measured behavior data of a pedestrian is compared. Thus, machine learning enables a more accurate inference of hearing impairment. In this way, the inference module 112 identifies pedestrians' hearing states that may negatively impact their safety such that appropriate countermeasures may be provided to reduce the likelihood of an unsafe environment surrounding the pedestrian.

Moreover, it should be appreciated that machine learning algorithms are generally trained to perform a defined task. Thus, the training of the machine learning algorithm is understood to be distinct from the general use of the machine learning algorithm unless otherwise stated. That is, the impaired hearing detection system 100 or another system generally trains the machine learning algorithm according to a particular training approach, which may include supervised training, self-supervised training, reinforcement learning, and so on. In contrast to training/learning of the machine learning algorithm, the impaired hearing detection system 100 implements the machine learning algorithm to perform inference. Thus, the general use of the machine learning algorithm is described as inference.

It should be appreciated that the inference module 112, in combination with the inference model 106, can form a computational model such as a neural network model. In any case, the inference module 112, when implemented with a neural network model or another model in one embodiment, implements functional aspects of the inference model 106 while further aspects, such as learned weights, may be stored within the data store 102. Accordingly, the inference model 106 is generally integrated with the inference module 112 as a cohesive, functional structure. Additional details regarding the machine-learning operation of the inference module 112 and inference model 106 are provided below in connection with FIG. 5.

The impaired hearing detection system 100 further includes a hearing test module 114 which, in one embodiment, includes instructions that cause the processor 108 to administer a hearing test to verify the hearing impairment of the pedestrian responsive to an inference of hearing impairment. That is, it may be that behavior data 104 and environment data 105 are inconclusive regarding hearing impairment or may lead to a false positive indication of hearing impairment. As such, the hearing test module 114 is the second stage of a multi-stage hearing impairment detection operation, which verifies the inference made by the first stage (i.e., the inference module 112). In other words, the output of the inference module 112 that a pedestrian may be experiencing hearing impairment is transmitted to the hearing test module 114, which administers a hearing test to confirm or refute the inference.

In an example, the hearing test module 114 transmits a command via the communication system 118 to the user device of the pedestrian to administer the hearing test. In a specific example, the hearing test module 114 includes instructions that cause the processor 108 to present an instruction regarding the administration of the hearing test. That is, the hearing test module 114 may generate a communication or notification to the pedestrian to take the hearing test. In an example, the notification may be haptic/tactile and/or visual, as an auditory notification may not be acknowledged due to the temporary hearing impairment. The notification may also recommend that the pedestrian stop in a safe place to take the hearing test to not distract a hearing-impaired test taker. In one particular example, the notification may indicate examples of safe/quiet places where the test may be taken and/or indicate a safe/quiet space near the pedestrian where the test may be taken.

In general, the hearing test may take a variety of forms and measures whether the hearing impairment of the pedestrian is greater than a threshold amount, which threshold amount may determine whether the pedestrian is exposing themselves and others to increased risk or not. In one particular example, the hearing test may quantify the degree of hearing impairment. In either case, the outcome of the hearing test may trigger a remedial countermeasure.

In an example, the hearing test may include producing, at a speaker of the user device, a sequence of tones varying in intensity (e.g., frequency or volume). The intensity of presented tones may increase or decrease as the test progresses. The test may prompt the pedestrian to indicate tones they can hear and cannot. That is, via a human interface element (such as a touch screen, icon, or physical button), the pedestrian may indicate which tone of the sequence of tones they have detected. Based on the frequency or volume of the tones that the pedestrian hears, the hearing test module 114 evaluates hearing impairment. For example, hearing impairment may be determined based on the quietest (measured in decibels) tone the pedestrian hears. If the quietest tone a pedestrian hears is louder than a threshold tone, which threshold tone may define a threshold hearing level to ensure pedestrian safety, the hearing test module 114 may confirm that the pedestrian is experiencing hearing impairment. By comparison, if the quietest tone a pedestrian hears is quieter than the threshold tone, the hearing test module 114 may invalidate the inference and conclude that the pedestrian is not experiencing hearing impairment. As such, if the pedestrian does not hear a threshold tone having a threshold intensity, the hearing test module 114 may confirm that the pedestrian is experiencing hearing loss, and the impaired hearing detection system 100 may perform a countermeasure as described below. In an example, the threshold tone may be user-defined based on a preference for when remedial countermeasures are to be applied or established by a manufacturer, engineer, or audiologist based on certain medical guidelines.

In an example, the hearing test is periodically re-administered following an initial indication of hearing impairment. That is, initially the hearing test may be triggered by an inference of hearing impairment. Once hearing impairment is verified, the hearing test module 114 may periodically re-administer the hearing test to determine when to conclude a particular countermeasure. For example, the countermeasure may be a recommendation to the pedestrian to remain in a location to avoid increasing the risk of danger based on moving in a hearing-impaired state. In this example, the recommendation to remain stationary may be removed when the pedestrian indicates that they can hear the threshold tone having the threshold frequency and/or volume.

As such, the hearing test module 114 provides a multi-stage modality to determine hearing impairment. As such, hearing impairment detection is improved by performing a confirming operation in the detection cycle.

The impaired hearing detection system 100 further includes a countermeasure module 116 which, in one embodiment, includes instructions that cause the processor 108 to produce a pedestrian assistance countermeasure responsive to verified hearing impairment for the pedestrian as determined from the hearing test. That is, the countermeasure module 116 may be communicatively coupled to the hearing test module 114 to receive a hearing test result.

As described above, safe navigation of busy streets, intersections, and other roadway infrastructure elements depends on a pedestrian's ability to perceive the environment accurately. Hearing impairment reduces the pedestrian's ability to perceive a component of that environment, specifically the sound environment. Given that the pedestrian may not accurately perceive the entire environment, the pedestrian may put themselves in danger when their hearing is impaired. The countermeasure module 116 may produce a countermeasure to offset or preclude the dangerous circumstances that may arise when a pedestrian is experiencing impaired hearing.

The pedestrian assistance countermeasure may take a variety of forms. In one example, the countermeasure may be a notification provided to the pedestrian via a user device of the pedestrian. For example, the countermeasure may be a message to the pedestrian to put on hearing protection, use a hearing aid device, consult a physician, or move away from the area with the high noise level. In another example, the recommendation could be to reduce the volume to make perception of ambient noise (such as audible safety cues and horns) easier.

In another example, the countermeasure may recommend that the pedestrian remains in a location. That is, it may be the case that encouraging a pedestrian to move may increase the danger to the pedestrian as such movement may be without the benefit of a full appreciation of the environment (i.e., the pedestrian does not assimilate the soundscape or sound environment). As such, the countermeasure may recommend that the user 1) remains in place and 2) utilize some hearing protection. In this example, the countermeasure module 116 may transmit a message to the user device via the communication system 118.

In another example, the countermeasure may include changing the operation of the user device. For example, the countermeasure module 116 may prevent further hearing impairment by activating a noise-canceling mode of the user device if the pedestrian is experiencing hearing impairment. As such, the countermeasure module 116 may cause the processor 108 to generate a notification or change the operation of the user device.

In addition to notifying the pedestrian, the countermeasure module 116 may generate a notification for other entities near the pedestrian. For example, the countermeasure module 116 may generate a notification to a human vehicle operator, an autonomous vehicle system, or an infrastructure element. These notifications may apprise the respective party/element of the presence of the impaired pedestrian so certain remedial actions can be administered to protect the pedestrian and others in the vicinity of the pedestrian. For example, a notification may be provided to a human vehicle operator so that the operator may slow down their vehicle to avoid any dangerous circumstances. Again, such notification may be transmitted to the human vehicle operator user device, manually-operated vehicle interface, autonomous vehicle system, or infrastructure element via the communication system 118 of the impaired hearing detection system 100.

As such, the present hearing impairment detection system 100 generates notifications that otherwise would not be generated, which notifications may be based on machine-learning evaluation of an environment. In this way, the pedestrian and surrounding individuals are apprised of hearing-impaired pedestrians that they would otherwise be unaware of.

In addition to notifying the entities in the vicinity of the pedestrian of the pedestrian's impaired hearing, the countermeasure module 116, in some examples, includes instructions that cause the processor 108 to produce a command signal for at least one of a vehicle in a vicinity of the pedestrian or an infrastructure element in the vicinity of the pedestrian. That is, as vehicles and infrastructure elements come within a threshold distance of the pedestrian, a communication path, such as a vehicle-to-pedestrian (V2P) or pedestrian-to-infrastructure (V2I) communication path, may be established between the impaired hearing detection system 100 and vehicles and infrastructure elements. In this example, the network membership may change based on the movement of the vehicles and pedestrians. In any event, via this network and the communication system 118 link between the impaired hearing detection system 100 and the entities of the cloud-based environment, command signals may be transmitted to the various entities, which command signals control the operation of the respective device to increase pedestrian/motorist safety. As a particular example, a command signal to a vehicle in the vicinity of the pedestrian may instruct the vehicle to decrease its speed when in the vicinity of the pedestrian. As another example, the command signal may generate a notification of the pedestrian on a digital billboard. While particular reference is made to particular command signals, other command signals may be generated by the countermeasure module 116. Additional examples are provided below in connection with FIG. 2. In any example, the command signal is transmitted to the respective entity via the communication system 118.

As such, the countermeasure module 116 improves vehicle perception of the surrounding environment by apprising the vehicle or driver of hearing-impaired pedestrians. Moreover, the countermeasure module 116 may improve vehicle control by determining vehicle operations based on detected hearing-impaired pedestrians in the vicinity of the vehicle.

As such, the impaired hearing detection system 100 of the present specification collects pedestrian behavior data 104 and compares such to baseline behavior to infer when the user may be in an impaired hearing state. Responsive to an inferred hearing-impaired state, a hearing test is administered to the pedestrian to verify that the pedestrian is in a hearing-impaired state. Responsive to a verified hearing-impaired state, the impaired hearing detection system 100 produces a countermeasure to offset or preclude the dangerous circumstance created by the pedestrian's impaired hearing.

FIG. 2 depicts the impaired hearing detection system 100 aiding a pedestrian 220 experiencing hearing impairment. As described above, roadways and the adjacent infrastructure are populated by various moving entities, including pedestrians 220 and vehicles 224. An accurate perception of the environment ensures the safety of pedestrians 220 and motorists alike. As such, when perception is impaired, so too is the safety of the pedestrian 220. As a particular example, crosswalk indicators may emit noise to indicate to pedestrians 220 when it is safe to cross a road and also when the light is about to change color such that the pedestrian should clear the intersection before vehicles 224 start moving across the crosswalk. A pedestrian 220 who is hearing impaired, for example due to a noisy environment such as a construction site, may not be able to hear the audible indication that the traffic light is about to change color and, therefore, may be unaware that a vehicle 224 is about to cross their path. The present impaired hearing detection system 100 prevents this situation by identifying when the pedestrian is hearing impaired via a multi-stage hearing impairment test and notifies and/or controls the pedestrian 220, vehicles 224, and infrastructure elements to alleviate the dangerous conditions.

FIG. 2 depicts one particular environment, a road intersection, where pedestrian/motorist safety may be particularly vulnerable. As depicted in FIG. 2, the pedestrian 220 is adjacent to a noisy environment (i.e., a construction site) while talking on the phone. The user is covering their car to block out the noise and is pacing to find a location where the noise may not interfere as much with their conversation. As described above, these movements may be detected by the user device 222, such as a phone, or another device, such as a personal health monitoring device worn by the pedestrian 220, and stored in the data store 102. As an additional example, cameras on the user device 222, dash cameras on a vehicle 224, or cameras on an infrastructure element 226 may further capture images of the pedestrian 220 from which movements of the pedestrian 220 may be determined. As described above, the inference module 112 may infer that a pedestrian 220 is experiencing hearing impairment based on this movement data.

Moreover, as described above, the behavior data 104 may include conversation data recorded by a microphone of the user device 222. This conversation data may also indicate that the pedestrian 220 is experiencing hearing impairment. As described above, the impaired hearing detection system 100 may collect behavior data 104 from the user device 222 of the pedestrian 220 and infer whether or not the pedestrian 220 is experiencing hearing impairment.

In one example, the noisy environment may trigger the activation of the inference module 112. That is, in one example, the inference module 112 continuously monitors the environment data (i.e., intensity and/or volume of detected sounds) and behavior data 104 to infer when the pedestrian 220 may be experiencing hearing impairment. In another example, a noisy environment may trigger the analysis of the behavior data 104 and environment data 105. For example, the user device 222 may include a microphone or other sound level monitoring device that continuously or periodically monitors the intensity of detected sounds. In this example, if a detected sound is greater than a threshold intensity (such as 85 decibels (dB) or 95 dB) for greater than a threshold period (e.g., 1 second, 10 seconds, 1 minute, etc.), the inference module 112 may be activated to analyze the behavior data 104 and/or the environment data 105. That is, in this example, the impaired hearing detection system 100 includes an instruction that causes the processor 108 to evaluate the sound environment of the pedestrian 220 and trigger inference of hearing impairment responsive to the sound environment having greater than a threshold intensity.

In addition to evaluating behavior data 104, the inference module 112 may evaluate environmental conditions surrounding the pedestrian that affect hearing impairment. The environmental conditions may come in various forms and be stored in the data store 102 as environment data 105. As described above, environment data 105 may indicate whether or not the pedestrian is in an environment where loud noises are expected. This environment data 105 may be weighted as described above, with environments indicative of loud sounds being more heavily weighted when determining that a pedestrian 220 is experiencing hearing impairment.

As described above, responsive to a determination that the pedestrian is experiencing hearing impairment, as inferred by the inference module 112 and verified by the hearing test module 114, the countermeasure module 116 produces any number of countermeasures that promote the safety of the pedestrian 220 and others in the environment. In some examples, the countermeasure is a notification, warning, alert, or command signal transmitted to the user device 222 based on the pedestrian's determined impaired decision-making state. The notification, warning, or alert may be transmitted to the user device 222, a vehicle 224, or an infrastructure element 226.

In an example, the notification transmitted to the user device 222 of the pedestrian 220 may include instructions to the pedestrian 220. For example, the impaired hearing detection system 100 may send an alert to the user device 222, directing the pedestrian 220 to remain stationary until the hearing test indicates that the pedestrian 220 can hear a threshold tone.

As a specific example, the impaired hearing detection system 100 may alert vehicles and other pedestrians that they are near/approaching an impaired pedestrian 220 through infrastructure elements such as digital billboards, external monitors on cars, mobile devices, traffic lights, etc. In one example, the impaired hearing detection system 100 may, via an augmented reality (AR) windshield, draw the driver's attention to the pedestrian by highlighting the pedestrian in the AR display.

As described above, the countermeasure may be a command signal transmitted to a vehicle 224, which command signal changes the operation of the vehicle 224 responsive to an identified pedestrian 220 with impaired hearing. Examples of operational changes triggered by the command signal include, but are not limited to 1) decreasing the vehicle 224 speed in a vicinity of the pedestrian 220, 2) increasing a volume of vehicle 224 horns, 3) modifying a braking profile of an automated vehicle 224 to be softer (i.e., brake sooner and more slowly), 4) modifying an acceleration profile of an automated vehicle 224 to be softer (i.e., accelerate more slowly and over a longer distance), 5) allowing for extra space between the vehicle 224 and the pedestrian 220, 6) rerouting the vehicle 224 to avoid being in the vicinity of the pedestrian 220, 7) increasing a clearance sonar sensitivity in the presence of the pedestrian 220, 8) turning off lane departure alerts in the vicinity of the pedestrian 220, 9) increasing adaptive cruise control distance setting to allow for more space between vehicles 224, 10) flashing lights at a pedestrian 220 to catch the attention of the pedestrian 220 to alter their decision-making state or encourage certain behavior (e.g., crossing a street), 11) turning down music in the cabin, 12) applying external one-way blackout to windows to prevent pedestrian from seeing inside the vehicle 224 thus simplifying the visual load on the pedestrian 220, 13) turning off non-safety related lights and or sounds to reduce the sensory load of the pedestrian 220, 14) rolling up windows to block out vehicle 224 cabin noise from further distracting/stressing the pedestrian 220, and 15) increasing a frequency of audible alerts or increase conspicuity of signals to increase chance of pedestrian 220 perception.

Moreover, as described above, the countermeasure may be a command signal transmitted to an infrastructure element 226, such as a traffic light. Examples include 1) repeating alerts or increasing the conspicuity of signals to increase the chance of pedestrian 220 perception, 2) altering signals to reroute traffic away from the pedestrian 220, 3) allowing extra time for the pedestrian 220 to cross at signaled intersections, and 4) turning off traffic signals when no vehicles 224 exist within a defined proximity. While particular reference is made to particular countermeasures, various countermeasures may be implemented to reduce or preclude the events that may arise due to a pedestrian's impaired decision-making state.

FIG. 3 depicts the impaired hearing detection system 100 inferring hearing impairment based on conversation data 328 of the pedestrian 220 and another participant 330 in a noisy environment. As described above, conversation data 328 may be indicative of impaired hearing. Not only are the conversation characteristics of the pedestrian 220 indicative of impaired hearing, but so are the conversation characteristics of a non-pedestrian participant 330 in the conversation. For example, as depicted in FIG. 3, the pedestrian 220 uttering a phrase such as “I'm sorry, can you speak up please?” may indicate that the pedestrian 220 is experiencing hearing impairment. Moreover, the non-pedestrian participant 330 repeating what they said and increasing their volume may provide additional evidence that the pedestrian 220 is experiencing hearing impairment. As such, the inference module 112 includes instructions that cause the processor 108 to perform speech analysis of the conversation data 328 of the pedestrian 220 and from a non-pedestrian participant 330 in a conversation to support an inference of hearing impairment.

In an example, the inference module 112 can differentiate hearing impairment from pedestrian confusion based on the speech analysis. For example, a pedestrian 220 uttering the phrase “could you repeat that?” may indicate that the pedestrian 220 cannot hear the non-pedestrian participant 330 or that the pedestrian 220 does not understand what the non-pedestrian participant 330 is saying. This differentiation between impaired hearing and confusion may be based on the conversation data 328 and/or the environment data 105. For example, the behavior data 104 for the non-pedestrian participant 330 may indicate that the non-pedestrian participant 330 has a pattern of speaking quickly and quietly and may exhibit other patterns that make it difficult for users to understand what the the non-pedestrian participant 330 is saying. Accordingly, the inference module 112 may identify these communication behaviors (e.g., speaking quickly and quietly) preceding the phrase “could you repeat that?” by the pedestrian 220 as indicating that the pedestrian 220 is confused but perhaps does not suffer from hearing impairment. In other words, some conversational behaviors may cause a pedestrian 220 to utter phrases that would otherwise indicate impaired hearing but do not yield an inference of impaired hearing because of the context of the conversation. The impaired hearing detection system 100 of the present specification identifies this contextual information (e.g., conversational habits of a non-pedestrian participant 330 and/or environmental conditions) to distinguish between behaviors indicative of hearing impairment and those that are not.

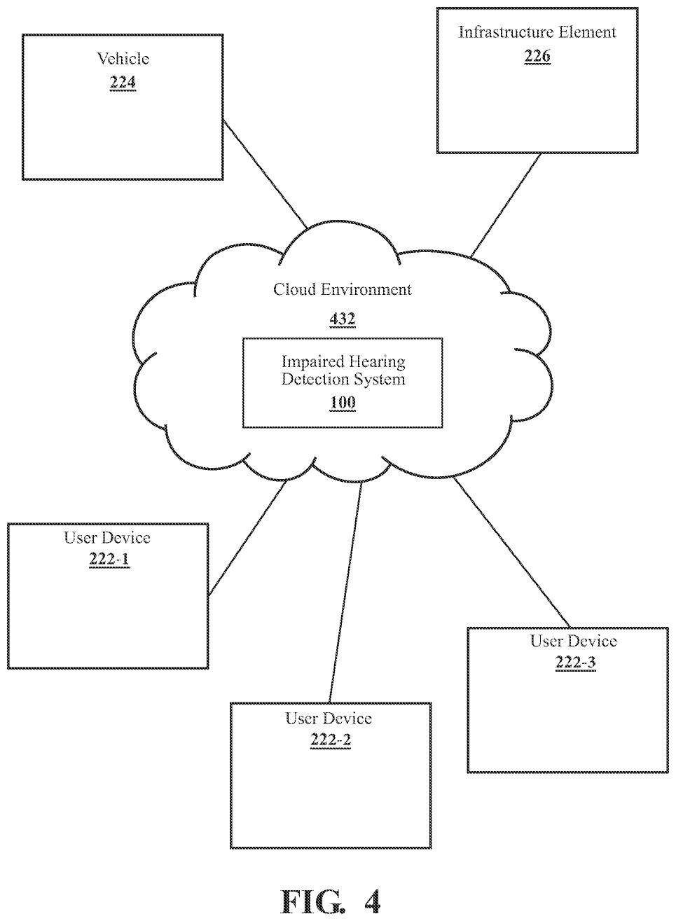

FIG. 4 illustrates one embodiment of the impaired hearing detection system of FIG. 1 in a cloud-computing environment 432. As illustrated in FIG. 4, in one example, the impaired hearing detection system 100 is embodied at least in part within the cloud-computing environment 432. The cloud-based environment 432 itself, as previously noted, is a dynamic environment that comprises cloud members who are routinely migrating into and out of a geographic area. In general, the geographic area, as discussed herein, is associated with a broad area, such as a city and surrounding suburbs. In any case, the area associated with the cloud environment 432 can vary according to a particular implementation but generally extends across a wide geographic area.

As described above, the impaired hearing detection system 100 includes a communication system 118 by which the impaired hearing detection system 100 can communicate with various entities to receive/transmit information to 1) infer pedestrian hearing impairment and 2) generate countermeasures that prevent dangerous situations that may arise due to the hearing impairment. Specifically, the impaired hearing detection system 100 communicates, via the communication system 118, with user devices 222-1, 222-2, 222-3 to 1) collect behavior data 104 characterizing a pedestrian 220 from which an inference of hearing impairment is made and 2) compile baseline data from the pedestrian 220 and additional users against which currently collected behavior data 104 for a pedestrian is compared. Moreover, the impaired hearing detection system 100 may communicate, via the communication system 118, with the vehicle 224 and/or infrastructure element 226 in the vicinity of the pedestrian 220 to collect movement data about the pedestrian 220. That is, the vehicles 224 and/or infrastructure elements 226 in the vicinity of the pedestrian 220 may include cameras that capture bodily movements, facial movements, and/or eye movements of pedestrians. This information is received and used by the inference module 112 to infer an impaired state of the hearing of the pedestrian 220. Accordingly, in one or more approaches, the cloud environment 432 may facilitate communications between multiple user devices 222-1, 222-2, 222-3, vehicles 224, and infrastructure elements 226 to acquire and distribute information from the user devices 222, vehicles 224, and infrastructure elements 226 to the impaired hearing detection system 100.

Still further, via the communication system 118, the impaired hearing detection system 100, and more specifically, the countermeasure module 116, may transmit notifications, messages, alerts, and/or command signals to the user devices 222 (of the pedestrian and other individuals), vehicles 224, and infrastructure elements 226. That is, via the communication system 118, the impaired hearing detection system 100 outputs the countermeasures generated by the countermeasure module 116.

As such, by collecting data from several users, those pedestrians exhibiting impaired hearing and would thus benefit from targeted assistance are identified and the target assistance provided. Such a system identifies potentially dangerous situations that may otherwise go unnoticed were behaviors not monitored to determine impaired hearing.

FIG. 5 illustrates one embodiment of a machine-learning impaired hearing detection system 100 associated with assisting pedestrians exhibiting impaired hearing. Specifically, FIG. 5 depicts the inference module 112, which in one embodiment with the inference model 106, administers a machine learning algorithm to generate a hearing impairment inference 438 for the pedestrian 220, which hearing impairment inference 438 triggers execution of a hearing test to verify the inference.

As described above, the machine-learning model may take various forms, including a machine-learning model that is supervised, unsupervised, or reinforcement-trained. In one particular example, the machine-learning model may be a neural network that includes any number of 1) input nodes that receive behavior data 104 and environment data 105, 2) hidden nodes, which may be arranged in layers connected to input nodes and/or other hidden nodes and which include computational instructions for computing outputs, and 3) output nodes connected to the hidden nodes which generate an output indicative of the hearing impairment inference 438 for the pedestrian 220.

As described above, the inference module 112 relies on baseline data to infer a hearing-impaired state of the pedestrian 220. Specifically, the inference module 112 may acquire baseline pedestrian data 434, stored as behavior data 104 in the data store 102, and baseline population data 436, which is also stored as behavior data 104 in the data store 102. The baseline data may be characterized as whether it represents impaired or unimpaired hearing. That is, the pedestrian 220 and other users may exhibit certain patterns when their hearing is unimpaired and others when their hearing is impaired. The baseline data may reflect both of these conditions, and the inference module 112, whether supervised, unsupervised, or reinforcement-trained, may detect similarities between the behaviors of the pedestrian 220 with the patterns identified in the baseline pedestrian data 434 and/or the baseline population data 436.

As an example, behavior data 104 may indicate that a pedestrian 220 is speaking with reduced word sharpness and increased word elongation than expected for the pedestrian 220 based on the baseline pedestrian data 434. In other words, the inference module 112, along with the inference model 106, compares currently identified behavior data 104 with what is typical or expected for the pedestrian 220 and/or other users, based on historically collected data and relies on a machine-learning inference model 106 to generate a hearing impairment inference 438 based on the comparison of the historically determined pedestrian/population patterns and the currently measured behavior data 104. Note that while a few examples of behavior data (i.e., decreased sharpness and increased elongation) are relied on in generating an inference, the inference module 112 may consider several different factors when generating an inference. That is, it may be that one characteristic by itself is not sufficient to infer a hearing-impaired state for a pedestrian 220 correctly. As such, the inference module 112 relies on multiple data points from both the behavior data 104 and the baseline data to infer the state of the pedestrian.

Note that in some examples, the machine-learning model is weighted to rely more heavily on baseline pedestrian data 434 than baseline population data 436. That is, while certain behaviors indicate impaired hearing, some users communicate in a way that deviates from the population behavior but does not constitute impaired hearing. For example, the pedestrian 220 may routinely walk with an elongated step length, speak more loudly than the general public, and produce facial movements that otherwise would indicate hearing impairment. Compared to the general population, this may be indicative of impaired hearing. However, given that it is the standard, or baseline, behavior for this particular pedestrian 220, these particular communication and movement behaviors may not indicate impaired hearing. As such, the inference module 112 may weigh the interaction patterns of the pedestrian more heavily than the interaction patterns of the additional individuals.

Moreover, it should be noted that the baseline pedestrian data 434 may change over time. For example, as users age, they may habitually speak more loudly. As such, the inference module 112 may include instructions that cause the processor 108 to update the machine-learning instruction set to compare the behavior data 104 of the pedestrian 220 to the baseline data based on continuously collected behavior data 104 for the pedestrian 220. As such, the inference 438 is robust against the changing behaviors of the pedestrian 220.

As stated above, the inference module 112 considers different deviations and generates an inference 438. However, as each deviation from baseline data may not conclusively indicate impaired hearing, the inference module 112 considers and weights different deviations when generating the inference 438. For example, as described above, the inference module 112 may consider the quantity, frequency, and degree of deviation between the behavior data 104 and the baseline data when generating the inference 438.

In any example, if the deviation is greater by some threshold than the baseline data, the inference module 112 outputs an inference 438, which inference 438 may be binary or graduated. For example, if the frequency, quantity, and degree of deviation surpass a threshold, the inference module 112 may indicate that the pedestrian 220 has hearing impairment. By comparison, if the frequency, quantity, and degree of deviation do not surpass the threshold, the inference module 112 may indicate that the pedestrian does not have hearing impairment. In another example, the output may indicate a degree of impaired hearing, which may be determined based on the frequency, quantity, and degree of deviation of the behavior data 104 from the baseline data.

In any case, the inferences 438 may be passed to the inference module 112 to refine the machine-learning algorithm. For example, a user may be prompted to evaluate the inference provided. This user feedback may be transmitted to the inference module 112 such that future inferences may be generated based on the correctness of past inferences. That is, feedback from the user or other source may be used to refine the inference module 112 to more accurately infer the pedestrian's hearing state based on measured behavior data 104.

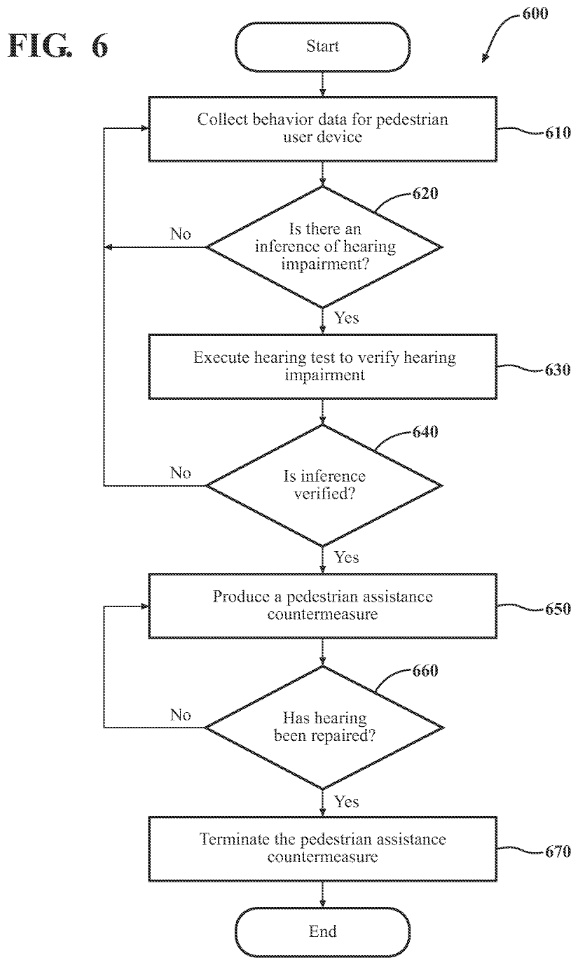

Additional aspects of alleviating impaired hearing-based pedestrian risks will be discussed in relation to FIG. 6. FIG. 6 illustrates a flowchart of a method 600 that is associated with identifying and verifying a pedestrian's hearing impairment and providing countermeasures accordingly. Method 600 will be discussed from the perspective of the impaired hearing detection system 100 of FIG. 1. While method 600 is discussed in combination with the impaired hearing detection system 100, it should be appreciated that the method 600 is not limited to being implemented within the impaired hearing detection system 100 but is instead one example of a system that may implement the method 600.

At 610, the impaired hearing detection system 100 collects behavior data 104 from the pedestrian user device 222. For example, the impaired hearing detection system 100 may communicate with multiple user devices 222 to establish baseline data and determine current behavior data 104 for a pedestrian 220. In an example, the impaired hearing detection system 100 acquires the behavior data 104 at successive iterations or time steps. Thus, the impaired hearing detection system 100, in one embodiment, iteratively administers the functions discussed at blocks 610-620 to acquire the behavior data 104 and provide information therefrom. Furthermore, the impaired hearing detection system 100, in one embodiment, administers one or more of the noted functions in parallel in order to maintain updated perceptions.

At 620, the inference module 112 infers, from the behavior data 104 and/or environment data 105 collected by a user device 222, whether the pedestrian 220 is experiencing hearing impairment based on a comparison with baseline data. As described above, the baseline data may include historical conversational patterns of the pedestrian 220 and/or other users (e.g., general population and/or regional population) and may further be classified as indicative of impaired or unimpaired behavior of the pedestrian 220 and/or other users. The baseline data represents expected or anticipated behavior for the pedestrian 220 based on their historical patterns and/or the historical patterns of additional users. In an example, the inference module 112 determines whether any deviation(s) between the currently measured behavior data 104 and the baseline data is greater or less than a threshold. If not greater than a threshold, then the impaired hearing detection system 100 continues to monitor behavior data 104.

If the deviation(s) is greater than a threshold, then at 630, the hearing test module 114 administers a hearing test to verify hearing impairment. As described above, an inference of hearing impairment alone may be insufficiently reliable to justify generating a notification and/or taking control of a vehicle 224 or infrastructure element 226. As such, a hearing test may be administered to verify the inference. As described above, the verification may include presenting a sequence of tones having increasing or decreasing frequency and/or loudness and determining the lowest frequency tone that the pedestrian 220 can hear. If the hearing test does not verify the inference of hearing impairment, 640, no, the impaired hearing detection system 100 returns to collecting behavior data 104.

If the hearing test does verify the inference, at 650, the countermeasure module 116 produces a pedestrian assistance countermeasure responsive to a verified hearing impairment of the pedestrian 220 as determined by the hearing test. As described above, such countermeasures may take various forms and may include a notification to the pedestrian, such as to wear hearing protection or remain stationary to avoid the danger that may come from a movement that is ignorant to sound-based warnings. In another example, the countermeasure may be a notification or a command signal transmitted to entities (e.g., vehicles, drivers, and infrastructure elements) in the vicinity of the hearing-impaired pedestrian to take remedial actions to reduce the danger resulting from the impaired hearing state of the pedestrian 220.

At 660, the system determines whether the hearing has been repaired. Specifically, the hearing test module 114 may periodically administer the hearing test to see if the pedestrian's 220 hearing has returned. For example, the hearing test module 114 may re-administer the hearing test to determine if the pedestrian 220 can hear the threshold tone. If not, the countermeasure module 116 maintains the generated countermeasure in place. If so, at 670, the countermeasure module 116 may terminate the pedestrian assistance countermeasure.

As such, the present system, methods, and other embodiments promote the safety of all road users by identifying pedestrians 220 who are experiencing hearing impairment based on their behavior (e.g., conversational behavior or movement behavior).

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-6, but the embodiments are not limited to the illustrated structure or application.

The flowcharts and block diagrams in the figures illustrate the architecture, functionality, and operation of possible implementations of systems, methods, and computer program products according to various embodiments. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be administered substantially concurrently, or the blocks may sometimes be administered in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. The systems, components and/or processes also can be embedded in a computer-readable storage, such as a computer program product or other data program storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. A non-exhaustive list of the computer-readable storage medium can include the following: a portable computer diskette, a hard disk drive (HDD), a solid-state drive (SSD), a read-only memory (ROM), an erasable programmable read-only memory (EPROM or Flash memory), a portable compact disc read-only memory (CD-ROM), a digital versatile disc (DVD), an optical storage device, a magnetic storage device, or a combination of the foregoing. In the context of this document, a computer-readable storage medium is, for example, a tangible medium that stores a program for use by or in connection with an instruction execution system, apparatus, or device.

Program code embodied on a computer-readable medium may be transmitted using any appropriate medium, including but not limited to wireless, wireline, optical fiber, cable, RF, etc., or any suitable combination of the foregoing. Computer program code for carrying out operations for aspects of the present arrangements may be written in any combination of one or more programming languages, including an object-oriented programming language such as Java™, Smalltalk, C++ or the like and conventional procedural programming languages, such as the “C” programming language or similar programming languages. The program code may administer entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The phrase “at least one of . . . and . . . ” as used herein refers to and encompasses any and all possible combinations of one or more of the associated listed items. As an example, the phrase “at least one of A, B, and C” includes A only, B only, C only, or any combination thereof (e.g., AB, AC, BC or ABC).

Aspects herein can be embodied in other forms without departing from the spirit or essential attributes thereof. Accordingly, reference should be made to the following claims, rather than to the foregoing specification, as indicating the scope hereof.

What is claimed is:

1. A system, comprising:
a processor; and
a memory storing machine-readable instructions that, when executed by the processor, cause the processor to:
infer that a pedestrian is experiencing hearing impairment based on data collected by a user device of the pedestrian;
administer a hearing test to verify the hearing impairment of the pedestrian responsive to an inference of hearing impairment; and
produce a pedestrian assistance countermeasure responsive to verified hearing impairment of the pedestrian as determined from the hearing test.

2. The system of claim 1, wherein the machine-readable instruction that, when executed by the processor, causes the processor to infer that the pedestrian is experiencing hearing impairment:
comprises a machine-readable instruction that, when executed by the processor, causes the processor to infer that the pedestrian is experiencing hearing impairment based on conversation data collected by a microphone of the user device; and
is a machine-learning instruction that performs speech analysis of the conversation data.

3. The system of claim 2, wherein:
the machine-learning instruction that, when executed by the processor, causes the processor to infer that the pedestrian is experiencing hearing impairment comprises a machine-readable instruction that, when executed by the processor, causes the processor to compare the conversation data associated with the pedestrian to baseline data to identify deviations therebetween; and
the baseline data comprises at least one of:
conversational patterns of the pedestrian; and
conversational patterns of additional users.

4. The system of claim 2, wherein the machine-learning instruction that, when executed by the processor, causes the processor to infer that the pedestrian is experiencing hearing impairment comprises a machine-readable instruction that, when executed by the processor, causes the processor to differentiate hearing impairment from pedestrian confusion based on the speech analysis.

5. The system of claim 4, wherein the machine-readable instruction that, when executed by the processor, causes the processor to differentiate hearing impairment from pedestrian confusion comprises a machine-readable instruction that, when executed by the processor, causes the processor to perform speech analysis of conversation data from a non-pedestrian participant in a conversation.

6. The system of claim 2, wherein the machine-learning instruction that, when executed by the processor, causes the processor to infer that the pedestrian is experiencing hearing impairment comprises a machine-readable instruction that, when executed by the processor, causes the processor to evaluate environmental conditions surrounding the pedestrian that affect hearing impairment.

7. The system of claim 1, wherein the machine-readable instruction that, when executed by the processor, causes the processor to produce the pedestrian assistance countermeasure comprises a machine-readable instruction that, when executed by the processor, causes the processor to generate a notification or change an operation of the user device.

8. The system of claim 1, wherein the machine-readable instruction that, when executed by the processor, causes the processor to administer the hearing test to verify the hearing impairment of the pedestrian comprises a machine-readable instruction that, when executed by the processor, causes the processor to:
produce a sequence of tones varying in at least one of frequency or volume;
receive an indication of user perception of a threshold tone of the sequence of tones; and
evaluate the hearing impairment based on the indication of user perception of the threshold tone.

9. The system of claim 1, wherein the machine-readable instruction that, when executed by the processor, causes the processor to administer the hearing test to verify the hearing impairment of the pedestrian comprises a machine-readable instruction that, when executed by the processor, causes the processor to present an instruction regarding an administration of the hearing test.

10. The system of claim 1, wherein the machine-readable instructions further comprise a machine-readable instruction that, when executed by the processor, causes the processor to:
evaluate a sound environment of the pedestrian; and
trigger the inference of hearing impairment responsive to the sound environment having greater than a threshold volume.

11. The system of claim 1, wherein the machine-readable instruction that, when executed by the processor, causes the processor to infer that the pedestrian is experiencing hearing impairment comprises a machine-readable instruction that, when executed by the processor, causes the processor to infer that the pedestrian is experiencing hearing impairment based on a physical movement of the pedestrian.

12. The system of claim 1, wherein the machine-readable instruction that, when executed by the processor, causes the processor to produce the pedestrian assistance countermeasure comprises a machine-readable instruction that, when executed by the processor, causes the processor to generate a notification to the pedestrian to remain stationary until the hearing test indicates that the pedestrian can hear a threshold tone.

13. The system of claim 1, wherein the machine-readable instruction that, when executed by the processor, causes the processor to produce the pedestrian assistance countermeasure comprises a machine-readable instruction that, when executed by the processor, causes the processor to produce a notification to at least one of:
a human vehicle operator;
an autonomous vehicle system; or
an infrastructure element.

14. A non-transitory machine-readable medium comprising instructions that, when administered by a processor, cause the processor to:
infer that a pedestrian is experiencing hearing impairment based on data collected by a user device of the pedestrian;
administer a hearing test to verify the hearing impairment of the pedestrian responsive to an inference of hearing impairment; and
produce a pedestrian assistance countermeasure responsive to verified hearing impairment of the pedestrian as determined from the hearing test.

15. The non-transitory machine-readable medium of claim 14, wherein the instruction that, when executed by the processor, causes the processor to infer that the pedestrian is experiencing hearing impairment:
comprises an instruction that, when executed by the processor, causes the processor to infer that the pedestrian is experiencing hearing impairment based on conversation data of at least one of the pedestrian and another participant in a conversation; and
is a machine-learning instruction that compares the conversation data to baseline data to identify deviations therebetween.

16. The non-transitory machine-readable medium of claim 14, wherein the instruction that, when executed by the processor, causes the processor to infer that the pedestrian is experiencing hearing impairment comprises a machine-readable instruction that, when executed by the processor, causes the processor to differentiate hearing impairment from pedestrian confusion based on speech analysis.

17. The non-transitory machine-readable medium of claim 14, wherein the instruction that, when executed by the processor, causes the processor to infer that the pedestrian is experiencing hearing impairment comprises an instruction that, when executed by the processor, causes the processor to infer that the pedestrian is experiencing hearing impairment based on a physical movement of the pedestrian.

18. A method, comprising:
inferring that a pedestrian is experiencing hearing impairment based on data collected by a user device of the pedestrian;
administering a hearing test to verify the hearing impairment of the pedestrian responsive to an inference of hearing impairment; and
producing a pedestrian assistance countermeasure responsive to verified hearing impairment of the pedestrian as determined from the hearing test.

19. The method of claim 18, wherein:
inferring that the pedestrian is experiencing hearing impairment comprises, using machine learning:
comparing conversation data associated with a conversation between the pedestrian and another user to baseline data; and
identifying deviations between the conversation data and the baseline data.

20. The method of claim 19, wherein inferring that the pedestrian is experiencing hearing impairment comprises differentiating hearing impairment from pedestrian confusion based on speech analysis of the conversation data.
